(19) United States
(12) Patent Application Publication
(10) Pub. No.: US 2017/0079693 A1
(43) Pub. Date: Mar. 23, 2017

(54) INTERSPINOUS PROCESS DEVICE

(71) Applicant: CHIN-BONE TECH. CO., LTD., Taipei (TW)

(72) Inventors: CHENG-KUNG CHENG, Taipei (TW); HSIN-CHANG CHEN, Taipei (TW); YU-SHU LAI, Taipei (TW)

(73) Assignee: CHIN-BONE TECH., CO., LTD., Taipei (TW)

(21) Appl. No.: 15/093,695

(22) Filed: Apr. 7, 2016

(30) Foreign Application Priority Data
Sep. 17, 2015 (TW) 104215047

(51) Int. Cl. A61B 17/70

(52) U.S. Cl. CPC A61B 17/7067; A61B 2017/00862

(57) ABSTRACT

An interspinous process device is disclosed, including an abutting member, two supporting members, and two fixing members, which are separated from each other and capable of being configured to an assembly. The abutting member has two ends and a central region for abutting upon a spinous process of a vertebra. Each of the supporting member is connected with the end of the abutting member and has a terminal end away from the abutting member. The fixing member has a connecting end for connecting with the terminal end of the supporting member and a fixing end for fixing on a vertebral arch of another vertebra. The interspinous process device disclosed herein provides a supporting force between the vertebra and the other vertebra.

### CROSS-REFERENCE TO RELATED APPLICATION

This application claims priority from Taiwan Patent Application No. 104215047, filed on Sep. 17, 2015, in the Taiwan Intellectual Property Office, the content of which are hereby incorporated by reference in their entirety for all purposes.

### BACKGROUND OF THE INVENTION

1. Field

The present invention relates to an interspinous process device, and especially relates to an interspinous process device assembled or disposed between vertebras of vertebration for supporting vertebration.

2. Description of the Related Art

Nowadays, herminated intervertebral disc (HIVD), also called intervertebral disc herniation or spinal disc herniation, is a common disease that people may suffer resulting from unhealthy lifestyle or bad life habits. HIVD may cause people's nerve to be compressed and have severe pain due to nerve compression. Accordingly, a variety of medical engineering techniques are provided to solve the problem mentioned above for decreasing the severe pain from nerve compression.

As disclosed in Taiwan Patent Application No. 201242565, a spinous process device is designed for providing good supporting performance. Referring to the description in the Taiwan Patent mentioned above, a first abutting portion and a second abutting portion are provided to respectively abut upon two ends of upper spinous process of a vertebra. In detail, the first abutting portion and the second abutting portion are fixed on two wing portions of a vertebral arch of the vertebra for providing upward supporting force for the spinous process device. Thus, a nerve compression status is released and severe pain due to the nerve compression is reduced.

However, the prior spinous process device mentioned above has many shortages. For example, when applying the prior spinous process device on people's vertebra, there may be a stress transmission between an upper spinous process of a vertebra and a lower spinous process of another vertebra (no matter how weak the probability of stress transmitting is). Further, the spinous process of the vertebra supported by the prior spinous process device may shift or move horizontally due to rotation of the vertebra, so that the first abutting portion and the second abutting portion may deviate from an optimal abutting location. Besides, other kinds of prior interspinous process devices are all assembled between the upper spinous process of a vertebra and the lower spinous process of another vertebra. The supporting force of these prior interspinous process devices may also transmit from the upper spinous process of the vertebra, the structure of the spinous process device itself, to the lower spinous process of another vertebra. That is, the supporting force may transmit along the structure of the interspinous process device. Thus, a spinous process of a vertebra may always have a risk of breaking or cracking caused by the stress transmission, and it accordingly results in injuring in interspinous ligament to effect the following surgical operation or following recovery treatment.

Furthermore, there is still a big problem that an implanting or assembling process for prior interspinous process device always needs to remove a portion of interspinous ligament and to cut off supraspinous ligament. Thus, people's ligaments are unavoidably injured or harmed seriously.

Therefore, the inventor(s) of the present invention meditate and design an interspinous process device for overcoming the problems of prior existing technique to obtain more versatile industrial applications and implementations.

### SUMMARY OF THE INVENTION

For the foregoing prior art problems, the object of the present invention is to provide an interspinous process device, which can overcome the problems caused by the prior art techniques mentioned above. In accordance with the object of the present invention, the interspinous process device comprises: an abutting member including two ends and a central region set between the two ends, wherein the abutting member abuts upon a spinous process of a vertebra by the central region; two supporting members connecting to the two ends of the abutting member respectively, wherein each of the supporting members has a terminal end away from the abutting member; and two fixing members, each fixing member correspondingly including a connecting end and a fixing end, wherein the connecting end is connected with the terminal end of the supporting member, and the fixing end is fixed on a vertebral arch of another vertebra.

In accordance with one preferred embodiments, the abutting member, the two supporting members, and the two fixing members are independent from each other, which can be combined or connected with each other. The abutting member and the supporting member can be connected by mortising, pivoting, screwing, binding, bonding, engaging, or magnetic force, etc.

In accordance with one preferred embodiments, the central region of the abutting member is bent corresponding to the fixing member to form a concave portion. That is, the central region of the abutting member is recessed from the fixing member. The abutting member abuts upon the spinous process of the vertebra by the concave portion, wherein the central region of the abutting member does not contact a spinous process of another vertebra directly.

In accordance with one preferred embodiments, the supporting member includes a curved segment and a straight segment. The supporting member is connected with the end of the abutting member on the curved segment, and the terminal end of the supporting member is on the straight segment.

In accordance with one preferred embodiments, the two fixing ends of the two fixing members are fixed on two vertebral arches of another vertebra respectively, and there are two fixing portions formed on the two vertebral arches of another vertebra respectively. The connecting end of the fixing member further includes a connecting hole for passing through and engaging the terminal end of the supporting member fixedly.

### BRIEF DESCRIPTION OF THE DRAWINGS

Features will become apparent to those of skill in the art by describing in detail exemplary embodiments with reference to the attached drawings in which:

FIG. 1 illustrates an explosion view of an interspinous process device according to a preferred embodiment of the present invention.

FIG. 2A illustrates a side view of an abutting member and supporting members of the interspinous process device according to a preferred embodiment of the present invention.

FIG. 2B illustrates a top view of an abutting member and supporting members of the interspinous process device according to another preferred embodiment of the present invention.

FIG. 3A illustrates a side view of the interspinous process device applied to a spinous process of a vertebra according to the preferred embodiment of the present invention.

FIG. 3B illustrates a top view of the interspinous process device applied to a spinous process of a vertebra according to the preferred embodiment of the present invention.

FIG. 3C illustrates a schematic of an assembling hole on an interspinous ligament for assembling the interspinous process device according to the preferred embodiment of the present invention.

FIG. 4 illustrates a schematic of an abutting member and supporting members of the interspinous process device according to another preferred embodiment of the present invention.

### DETAILED DESCRIPTION OF THE PREFERRED EMBODIMENTS

Example embodiments are described more fully hereinafter with reference to the accompanying drawings, one who skilled in the art can easily and clearly know and understand the functions and advantages of the present invention according to the following description. Furthermore, one who skilled in the art can embody and apply a variety of embodiments described hereinafter. However, since the drawings are shown for clearly describing and supporting the specification of the present invention, they may be not in real proportional scale or with precise arrangement. Therefore, they may be embodied in different forms and should not be construed as limited to the embodiments set forth herein. Rather, these embodiments are provided so that this disclosure will be thorough and complete, and will fully convey exemplary implementations to those skilled in the art.

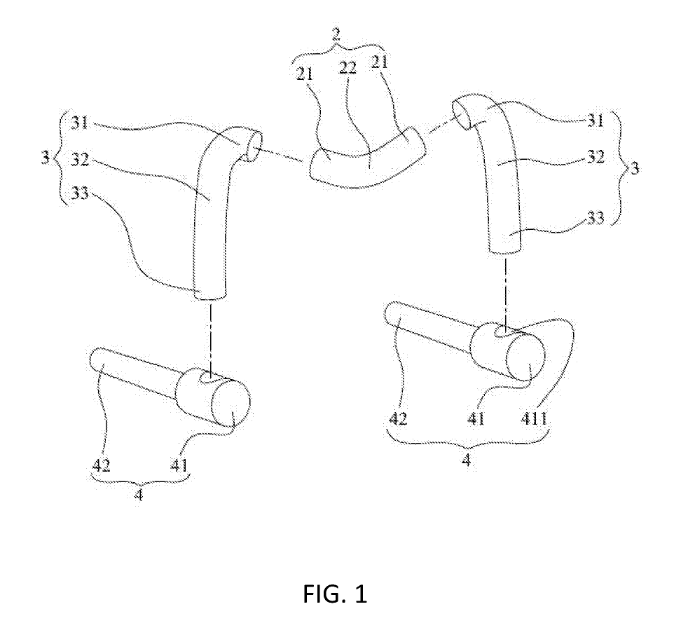

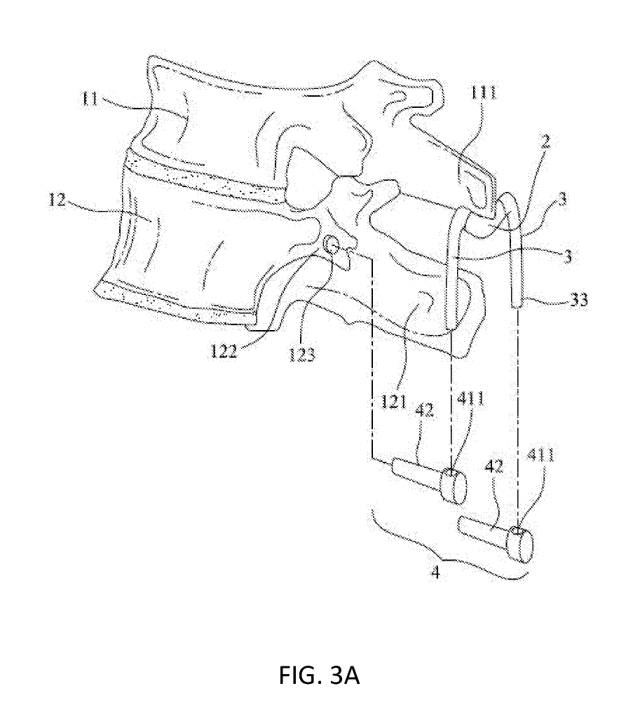

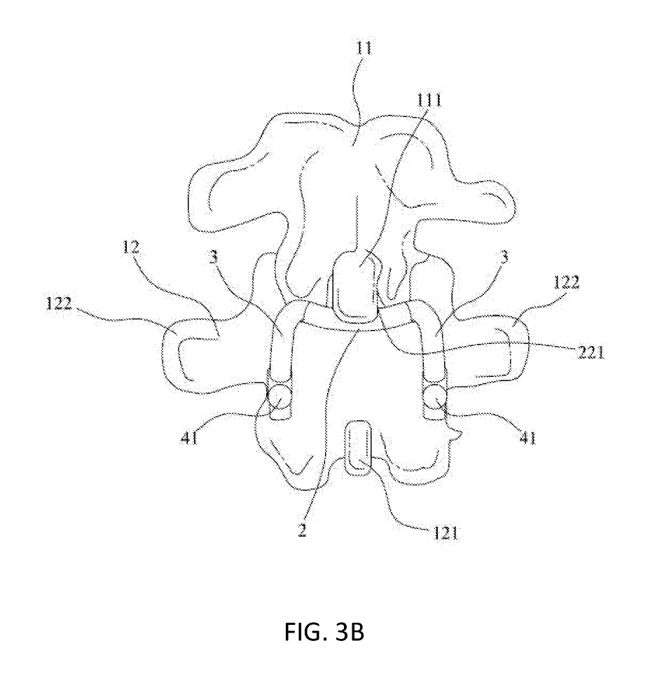

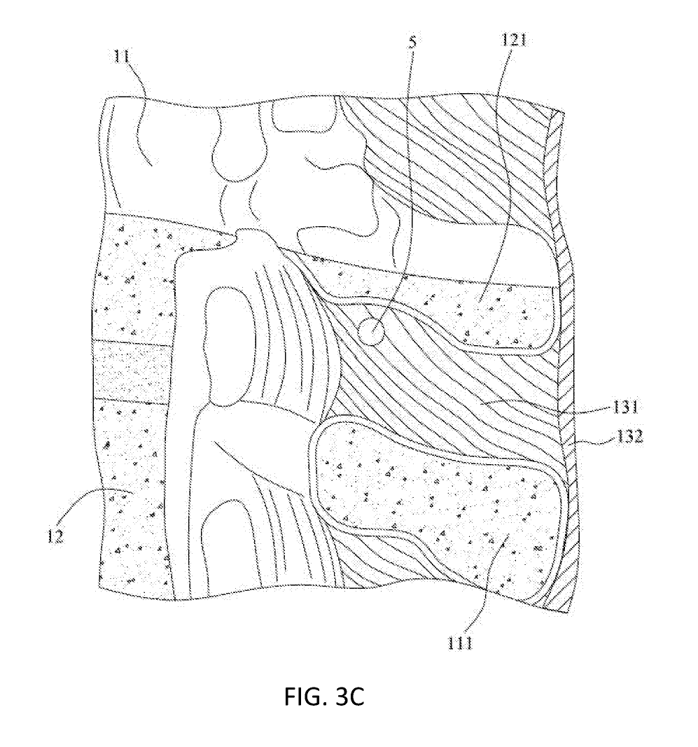

Referring to the related drawings, the following description will disclose some example embodiments of an interspinous process device of the present invention. For clarification and understanding, same elements are referred to same reference numerals throughout. Please refer to FIGS. 1, 3A and 3B, an interspinous process device of the present invention comprises: an abutting member 2, two supporting members 3 and two fixing members 4. The interspinous process device is assembled between a vertebra 11 and a vertebra 12. The abutting member 2, the two supporting members 3, and the two fixing members 4 are independent components, which can be combined or connected with each other. For example, the abutting member 2 and the supporting member 3 can be connected by mortising, pivoting, screwing, binding, bonding, or magnetic force, etc. The supporting members 3 and the fixing members 4 can be connected by bolting, mortising, pivoting, screwing, binding, bonding, engaging, or magnetic force, etc.

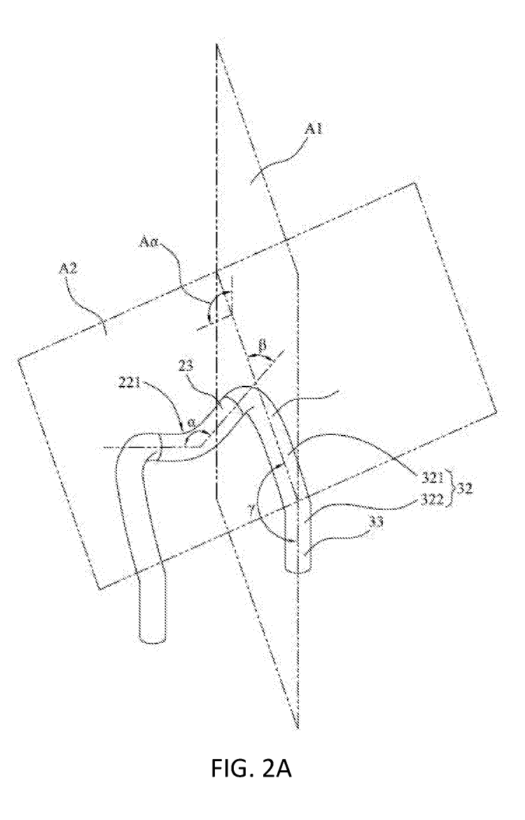

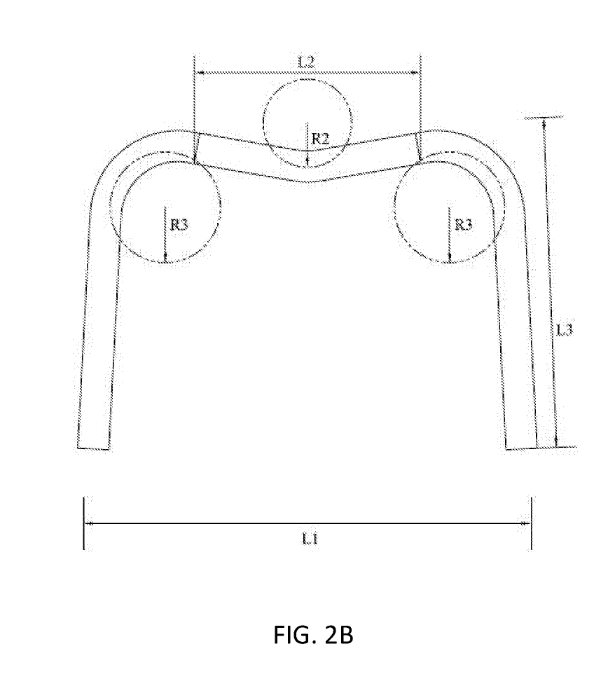

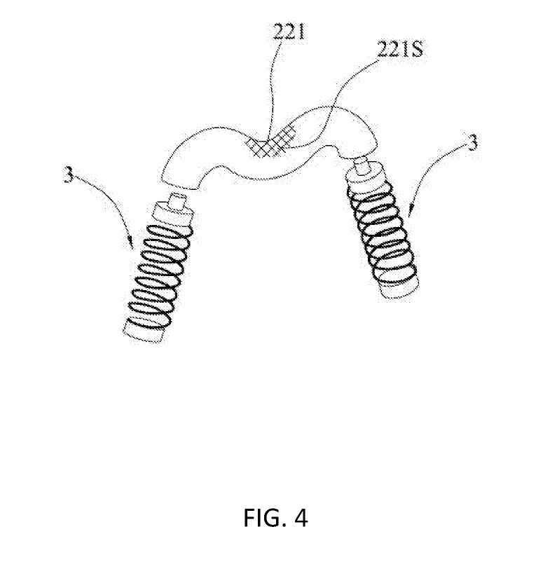

Referring to FIGS. 1, 2A-2B and 3A-3B, the abutting member 2 includes two ends 21 and a central region 22 set between the two ends 21. The central region 22 of the abutting member 2 is bent to the fixing member 4 to form a concave portion 221. That is, the concave portion 221 is formed by recessing the central region 22 from the fixing member 4. As shown in FIG. 3B, when the abutting member 2 abuts upon the spinous process 111 of the vertebra 11 by the concave portion 221, a portion of the spinous process 111 disposed within the concave portion 221, and the central region 22 of the abutting member 2 does not contact a spinous process 121 of the vertebra 12 directly. Further, as shown in FIG. 2A, the concave portion 221 of the abutting member 2 includes two extension segments 23, wherein the two extension segments 23 intersect at a location within the concave portion 221 with an included angle α in a range from 100 degrees to 170 degrees. Alternately, as shown in FIG. 2B, the shape of the concave portion 221 of the abutting member 2 can be in an arc shape belonging to a portion of a circumference of a circle, wherein the radius R2 of the circle is about 2 mm. The concave portion 221 of the abutting member 2 further includes a slip-resistant surface (as shown in FIG. 4), for providing a friction force when the abutting member 2 abuts upon the spinous process 111 of the vertebra 11. In accordance with the other preferred embodiments, the concave portion 221 can be made of a resilient material or a force-absorbing material. That is, a portion of the abutting member 2 or whole of the abutting member 2 can include a resilient material.

In accordance with the preferred embodiments, a structure diameter of the abutting member 2 is smaller than the distance between the spinous process 111 of the vertebra 11 and the spinous process 121 of the vertebra 12. The projection length L2 of the abutting member 2 is larger than the width of the spinous process 111 of the vertebra 11. The projection length L2 is preferred in a range from 10 mm to 60 mm.

As shown in FIGS. 1 and 2A-2C, two supporting members 3 are connected with the two ends 21 of the abutting member 2 respectively. The two supporting members 3 are substantially the same components with same dimensions, so that the lengths of the two supporting members are substantially equal. However, the lengths of the two supporting members 3 can be not equal in some specific situations. For example, in the situations of spine curvature, bone deformation, or relative bone position changed, the lengths of the two supporting members 3 can be corresponding not equal. The detailed structure of the supporting member 3 is described as follows. As shown in FIG. 1, the supporting member 3 includes a curved segment 31, a straight segment 32 and a terminal end 33. The curved segment 31 of the supporting member 3 is connected with the end 21 of the abutting member 2. The straight segment 32 is connected with the curved segment 31. The terminal end 33 is on one end of the straight segment 32 away from the abutting member 2. As shown in FIG. 2A, the extension segment 23 of the abutting member 2 and the straight segment 32 of the supporting member 3 have an included angle β in a range from 40 degrees to 90 degrees. As shown in FIG. 2B, the shape of the curved segment 31 of the supporting member 3 is in an arc shape belonging to a portion of a circumference of a circle, wherein the radius R3 of the circle is about 2.5 mm. Further, in accordance with the preferred embodiments, the straight segment 32 of the supporting member 3 includes a first sub-segment 321 and a second sub-segment 322 (shown in FIG. 2A), wherein the first sub-segment 321 and the second sub-segment 322 have an included angle γ in a range from 120 degrees to 170 degrees. The terminal end 33 of the supporting member 3 is on the second sub-segment 322 which is away from the abutting member 2. Generally speaking, as shown in FIG. 2A, a plane A1 composed of the first sub-segment 321 and the second sub-segment 322, and a plane A2 composed of the curved segment 31 is substantially perpendicular to each other. That is, an included angle A α between the plane A1 and the plane A2 is about 90 degrees. However, in accordance with the other embodiments, if it is necessary for integral assembling of the interspinous process device, and stress distribution and strength requirements fitted, the included angle A α between the plane A1 and the plane A2 can be not 90 degrees. For example, the included angle A α between the plane A1 and the plane A2 is preferred in a range from 50 degrees to 130 degrees.

As shown in FIG. 2B, the length L3 of the supporting member 3 is preferred larger than the distance between the spinous process 111 of the vertebra 11 and the spinous process 121 of the vertebra 12, wherein the length L3 of the supporting member 3 is preferred in a range from 10 mm to 60 mm. When the two supporting member 3 connect with the two ends 21 of the abutting member 2 respectively, a distance L1 between the terminal ends 33 of the two supporting members 3 is larger than the length L2 of the abutting member 2. The distance L1 between the two terminal ends 33 is preferred in a range from 25 mm to 60 mm. Further, the supporting member 3 includes a resilient structure, such as a spring, as shown in FIG. 4. Or, the supporting member 3 can be a buffer material, such as plastic material or rubber, etc (not shown in Figs.).

Further referring to FIGS. 1 and 3A-3B, the two fixing members 4 are connected with the two terminal ends 33 of the supporting members 3 respectively. The two fixing members 4 are substantially the same components with same dimensions, and the lengths of the two supporting members are substantially equal. However, the lengths of the two fixing members 4 can be not equal in some specific situations. For example, in the situations of spine curvature, bone deformation, or relative bone position changed, the lengths of the two fixing members 4 can be correspondingly not equal. While the lengths of the two fixing members 4 are not equal, a difference between the lengths of the two fixing members 4 is preferred to be less than 40 mm. In other words, the two fixing members 4 can adjust their lengths or other characteristics depending on different circumstances. The detailed structure of the fixing member 4 is described as follows. The fixing member 4 includes a connecting end 41 and a fixing end 42, wherein the connecting end 41 is connected with the terminal end 33 of the supporting member 3, and the fixing end 42 corresponds to the connecting end 41 and is used for fixing on a vertebral arch 122 of the vertebra 12. The connecting end 41 of the fixing member 4 further includes a connecting hole 411 for passing through and engaging the terminal end 33 of the supporting member 3 fixedly. There are further screw threads formed on the inner wall of the connecting hole 411, and there are corresponding screws formed on the surface of the terminal end 33 of the supporting member 3 for connecting the fixing member 4 with the supporting member 3 firmly. Furthermore, a fixing portion 123, such as a through hole, is further formed on the vertebral arch 122 of the vertebra 12 for fixing the fixing end 42 of the fixing member 4.

Continuously referring to FIGS. 3A-3B, they illustrate a side view and a top view of the interspinous process device applied between the vertebra 11 and the vertebra 12 according to the preferred embodiment of the present invention. The vertebra 11 is nearer a head of human body than the vertebra 12. After combining the abutting member 2, the two supporting members 3 and the two fixing members 4, the interspinous process device of the present invention is formed and can be assembled between the vertebra 11 and the vertebra 12. The two fixing members 4 are fixed on the vertebral arch 122 of the vertebra 12, and the abutting member 2 abuts upon the spinous process 111 of the vertebra 11, so that the interspinous process device can be assembled between the vertebra 11 and the vertebra 12 firmly. The interspinous process device of the present invention is assembled between the vertebra 11 and the vertebra 12 for providing a supporting force facing to the vertebra 12. The concave portion 221 of the abutting member 2 restrains an abutting region. Even if the vertebra 11 has an angle shifting in horizontal direction, the spinous process 111 of the vertebra 11 will be restrained within abutting region of the concave portion 221 of the abutting member 2 and does not leave away from the concave portion 221 of the abutting member 2. The supporting force provided by the interspinous process device of the present invention is mainly from the combined supporting members 3 and fixing members 4. Since the central region 22 of the abutting member 2 does not contact the spinous process 121 of the vertebra 12 directly, the stress does not transmit between the vertebra 11 and the vertebra 12.

In accordance with the preferred embodiments, the abutting member, the supporting members, and the fixing members are independent components, which can be combined or connected with each other by mortising, pivoting, screwing, binding, bonding, engaging or magnetic force, etc. The aforementioned modularized design of the present invention has many advantages; for example, easily-exchanging components. In addition, the interspinous process device of the present invention is suitable for a minimally invasive surgery, so that general surgical wounds within a length range from 8 cm to 15 cm according to prior art, can be decreased to smaller surgical wounds within a length range from 3 cm to 5 cm. Comparing with prior interspinous process device, the modularized interspinous process device of the present invention can be implanted into human body by opening or forming a small assembling hole 5 in an interspinous ligament 131 for assembling or accommodating the interspinous process device, as shown in FIG. 3C. Therefore, the ligament can be more protected that reduces injured opportunity, especially for the interspinous ligament 131 and a spine ligament 132. It is noted that, the assembling hole 5 according to the present invention is very small. For clearly describing the location of the assembling hole 5, please refer to FIG. 3C, which illustrates a schematic of the assembling hole on the interspinous ligament 131 exposed from partially opened spinous process 111 of the vertebra 11 and spinous process 121 of the vertebra 12.

As the above mentioned, the abutting member, the supporting members, and the fixing members of the present interspinous process device are independent components which can be combined with each other or be separated from each other. In addition to the aforementioned advantages that there is not any stress transmitting between the vertebra 11 and the vertebra 12 and a supporting force can be provided by combining the supporting members with the fixing members, the present interspinous process device can further provide a flexibility for assembling the abutting member, the supporting members, and the fixing members depending upon the different dimensions/requirements of patients or different circumstances. Therefore, every requirements and situations of patients for supporting force provided between a vertebra and another vertebra can be satisfied, and an assembling process of the present invention is more flexible than prior art.

The aforementioned embodiments have been disclosed herein, and although specific terms are employed, they are used and are to be interpreted in a generic and descriptive sense only and not for purpose of limitation. Accordingly, it will be understood by those of skill in the art that various changes in form and details may be made without departing from the spirit and scope of the present invention as set forth in the following claims.

What is claimed is:

1. An interspinous process device, comprising:
an abutting member including two ends and a central region for abutting upon a spinous process of a vertebra;
two supporting members, each of the supporting members being connected to the end of the abutting member and having a terminal end away from the abutting member; and
two fixing members, each fixing member correspondingly including a connecting end and a fixing end, wherein the connecting end is connected with the terminal end of the supporting member, and the fixing end is fixed on a vertebral arch of another vertebra.

2. The interspinous process device as claimed in claim 1, wherein the abutting member and the supporting member are connected by mortising, pivoting, screwing, binding, bonding, or magnetic force.

3. The interspinous process device as claimed in claim 1, wherein the central region of the abutting member is bent corresponding to the fixing member to form a concave portion, wherein that the concave portion of the abutting member abuts upon the spinous process of the vertebra, and the central region of the abutting member does not contact another spinous process of the vertebra directly.

4. The interspinous process device as claimed in claim 3, wherein the concave portion of the abutting member includes two extension segments, the two extension segments having an included angle in a range from 100 degrees to 170 degrees.

5. The interspinous process device as claimed in claim 3, wherein the shape of the concave portion of the abutting member is in an arc shape belonging to a portion of a circumference of a circle, wherein the radius of the circle is at least 2 mm.

6. The interspinous process device as claimed in claim 3, wherein the concave portion of the abutting member further includes a slip-resistant surface, and a portion of the abutting member or whole of the abutting member includes a resilient material.

7. The interspinous process device as claimed in claim 1, wherein the projection length of the abutting member is larger than the width of the spinous process of the vertebra, and a diameter of the abutting member structure is smaller than the distance between the spinous process of the vertebra and a spinous process of the another vertebra.

8. The interspinous process device as claimed in claim 1, wherein each of the supporting members includes a curved segment and a straight segment, wherein the supporting member is connected with the end of the abutting member by the curved segment, and the terminal end of the supporting member is on the straight segment.

9. The interspinous process device as claimed in claim 8, wherein the straight segment includes a first sub-segment and a second sub-segment, and the terminal end of the supporting member is on the second sub-segment, wherein a plane composed of the first sub-segment and the second sub-segment and a plane composed of the curved segment are substantially perpendicular to each other, or have an included angle in a range from 50 degrees to 130 degrees between each other, wherein the first sub-segment and the second sub-segment have an included angle in a range from 120 degrees to 170 degrees between each other.

10. The interspinous process device as claimed in claim 8, wherein the central region of the abutting member has a concave portion including two extension segments, and one of the two extension segments of the abutting member and the straight segment of the supporting member have an included angle in a range from 40 degrees to 90 degrees between each other.

11. The interspinous process device as claimed in claim 8, wherein the shape of the curved segment of the supporting member is in an arc shape belonging to a portion of a circumference of a circle, wherein the radius of the circle is at least 2.5 mm.

12. The interspinous process device as claimed in claim 1, wherein the lengths of the two supporting members are equal or not equal.

13. The interspinous process device as claimed in claim 1, wherein the supporting member includes a resilient structure.

14. The interspinous process device as claimed in claim 1, wherein the two fixing ends of the two fixing members are fixed on two vertebral arches of the another vertebra respectively, to form two fixing portions on the two vertebral arches of the another vertebra respectively.

15. The interspinous process device as claimed in claim 1, wherein the connecting end of the fixing member includes a connecting hole for passing through and engaging the terminal end of the supporting member fixedly.

16. The interspinous process device as claimed in claim 1, wherein the lengths of the two fixing members are equal or not equal, wherein while the lengths of the two fixing members are not equal, a difference between the lengths of the two fixing members is less than 40 mm.
